(19) United States
(12) Patent Application Publication
(10) Pub. No.: US 2020/0075177 A1
(43) Pub. Date: Mar. 5, 2020

(54) CONSTRUCTING THRESHOLDS BASED ON TEMPORAL PROFILES TO DETERMINE OUTLIERS IN SMART ENVIRONMENTS

(71) Applicant: International Business Machines Corporation, Armonk, NY (US)

(72) Inventors: Krishnasuri Narayanam, Bangalore (IN); Ramasuri Narayanam, Andhra Pradesh (IN); Mukundan Sundararajan, Bangalore (IN)

(21) Appl. No.: 16/121,256

(22) Filed: Sep. 4, 2018

(51) Int. Cl. G16H 80/00; G16H 50/30

(52) U.S. Cl. CPC G16H 80/00; G16H 50/30

(57) ABSTRACT

Determining outlier activities of daily living and instrumental activities of daily living and performing automatic actions in coordination with user inputs in smart environments. Threshold based activities of daily living temporal profiles constructed for a central processing hub of a smart home environment aid in distinguishing outlier activities from the possible exceptions that could occur during the execution of activities of daily living and instrumental activities of daily living.

### BACKGROUND

The present invention relates to determining outliers for activities of daily living, and more specifically to constructing thresholds based on temporal profiles associated with activities of daily living to determine outliers for activities of daily living. Automatic action in coordination with the user inputs in a smart environment can be performed based on the outlier activities of daily living.

Health monitoring is important for individuals to determine an amount of assistance which may be needed to maintain the health of a user. Health monitoring can be achieved through monitoring activities of daily living. Activities of daily living (ADL) refer to people's daily self-care activities. Basic ADLs include, but are not limited to: self-feeding, walking (ambulating), dressing and grooming, bathing, toileting, and transferring (moving from one body position to another). Self-Feeding ADL can include whether a user can feed themselves with or without cutlery. The bathing ADL can including showering and washing. The dressing and grooming ADL can include brushing/combing/styling hair, cleaning teeth, selecting appropriate clothes, putting said clothes on. The toileting ADL can include getting to the toilet, cleaning oneself, and getting back up. The transferring ADL can include walking, get in and out of bed, and get into and out of a chair or moving from one place to another while performing activities.

Other activities such as Instrumental Activities of Daily Living (IADL) are activities performed by an individual on a day to day basis that are not essential to basic self-care and independent living, but add quality to the way of life. These activities are not indispensable to a person's survival and fundamental functioning, but they do let someone live independently in society and function well as a self-reliant individual.

The repeated failure of a person in performing IADLs is usually a precursor to assisted living (at least in part) be it home care or the admission of the person to an assisted living and care facility. Examples of IADLs are: being able to do housework and prepare meals; taking prescribed medicines and keeping track of physician visits; managing money; shopping; using the telephone/computer as a means of communication; managing transportation; managing the household in its entirety (taking care of pets); and extracurricular activities.

Manual tracking of ADLs and IADLs is time consuming and often inaccurate.

A smart environment is an environment in which a variety of smart devices of different types including tags, sensors and controllers and have different form factors ranging from nano- to micro- to macro-sized. Virtual computing environments enable smart devices to access pertinent services anywhere and anytime. Smart devices can include mobile phones, surface-mounted devices (wearable computing) and embedded devices.

Rules can be designed to identify ADLs and IADLs within a smart environment. Rules can contain a start time interval for an ADL, time taken by the ADL, whether the ADL is regular and outlier behavior. The start time of an ADL refers to the time interval to which a usual start time of the ADL belongs. The time taken by the ADL refers to the typical amount of time an ADL takes. The regular ADL classification is for ADLs that happen repetitively within a time period. Outlier behavior is considered present if either the time taken by the ADL exceeds a typical time of execution (Type 1 outlier) or the ADL is not carried out after passing a typical time of execution (Type 2 outlier).

For example, data from multiple sensors present in a stove, refrigerator, mixer and faucet can imply that cooking is underway. It is possible that multiple rules can inference the same ADL. In a smart environment, multiple sensors are required to monitor and determine whether a user is completing an ADL or if the outlier behavior relative to the ADL is being exhibited.

### SUMMARY

According to one embodiment of the present invention, a method of determining outlier activities of a user relative to daily living activities or instrumental daily living activities in a smart environment is disclosed. The smart environment comprising a plurality of sensors in communication with a computer. The method comprising the steps of: the computer receiving sensor data from the plurality of sensors in the smart environment; the computer applying the sensor data to rules defining activities of daily living and instrumental activities of daily living to collect a corpus of activities of daily living and instrumental activities of daily living of the user; the computer constructing an activity of daily living and instrumental activity of daily living temporal profile based on the corpus of activities associated with the user; the computer setting a threshold for each activity of daily living and instrumental activity of daily living temporal profile associated with the user; the computer activating and monitoring the plurality of sensors associated with activity of daily living and instrumental activity of daily living during a distribution of start times associated the activity of daily living and instrumental activity of daily living temporal profiles to determine outliers and exceptions of the activities of daily living and instrumental activities of daily living associated with the user above the threshold; the computer, for each outlier, sending an alert to at least the user regarding the outlier with associated actions within the smart environment; and the computer receiving chosen associated actions from the user and implementing the associated actions within the smart environment.

According to another embodiment of the present invention, a computer program product for determining outlier activities of a user relative to daily living activities or instrumental daily living activities in a smart environment. The smart environment comprising a plurality of sensors in communication with a computer comprising at least one processor, one or more memories, one or more computer readable storage media, the computer program product comprising a computer readable storage medium having program instructions embodied therewith. The program instructions executable by the computer to perform a method comprising: receiving, by the computer, sensor data from the plurality of sensors in the smart environment; applying, by the computer, the sensor data to rules defining activities of daily living and instrumental activities of daily living to collect a corpus of activities of daily living and instrumental activities of daily living of the user; constructing, by the computer, an activity of daily living and instrumental activity of daily living temporal profile based on the corpus of activities associated with the user; setting, by the computer, a threshold for each activity of daily living and instrumental activity of daily living temporal profile associated with the user; activating and monitoring, by the computer, the plurality of sensors associated with activity of daily living and instrumental activity of daily living during a distribution of start times associated the activity of daily living and instrumental activity of daily living temporal profiles to determine outliers and exceptions of the activities of daily living and instrumental activities of daily living associated with the user above the threshold; for each outlier, sending, by the computer, an alert to at least the user regarding the outlier with associated actions within the smart environment; for each exception, implementing the associated actions within the smart environment based on predefined actions; and receiving, by the computer, chosen associated actions from the user and implementing the associated actions within the smart environment.

According to another embodiment of the present invention, a computer system for determining outlier activities of a user relative to daily living activities or instrumental daily living activities in a smart environment. The smart environment comprising a plurality of sensors in communication with a computer. The computer system comprising the computer comprising at least one processor, one or more memories, one or more computer readable storage media having program instructions executable by the computer to perform the program instructions. The program instructions comprising: receiving, by the computer, sensor data from the plurality of sensors in the smart environment; applying, by the computer, the sensor data to rules defining activities of daily living and instrumental activities of daily living to collect a corpus of activities of daily living and instrumental activities of daily living of the user; constructing, by the computer, an activity of daily living and instrumental activity of daily living temporal profile based on the corpus of activities associated with the user; setting, by the computer, a threshold for each activity of daily living and instrumental activity of daily living temporal profile associated with the user; activating and monitoring, by the computer, the plurality of sensors associated with activity of daily living and instrumental activity of daily living during a distribution of start times associated the activity of daily living and instrumental activity of daily living temporal profiles to determine outliers and exceptions of the activities of daily living and instrumental activities of daily living associated with the user above the threshold; for each outlier, sending, by the computer, an alert to at least the user regarding the outlier with associated actions within the smart environment; and receiving, by the computer, chosen associated actions from the user and implementing the associated actions within the smart environment.

### BRIEF DESCRIPTION OF THE DRAWINGS

FIG. 1 depicts an exemplary diagram of a possible data processing environment in which illustrative embodiments may be implemented.

FIG. 2 illustrates internal and external components of a client computer and a server computer in which illustrative embodiments may be implemented.

FIG. 3 shows a flow diagram of a method of determining outlier ADLs and performing automatic actions in coordination with user inputs in smart environments.

### DETAILED DESCRIPTION

It is to be understood that although this disclosure includes a detailed description of data processing environment, the implementation of the teachings recited herein are not limited to such an environment and can be implemented within a cloud computing environment. Rather, embodiments of the present invention are capable of being implemented in conjunction with any other type of computing environment now known or later developed.

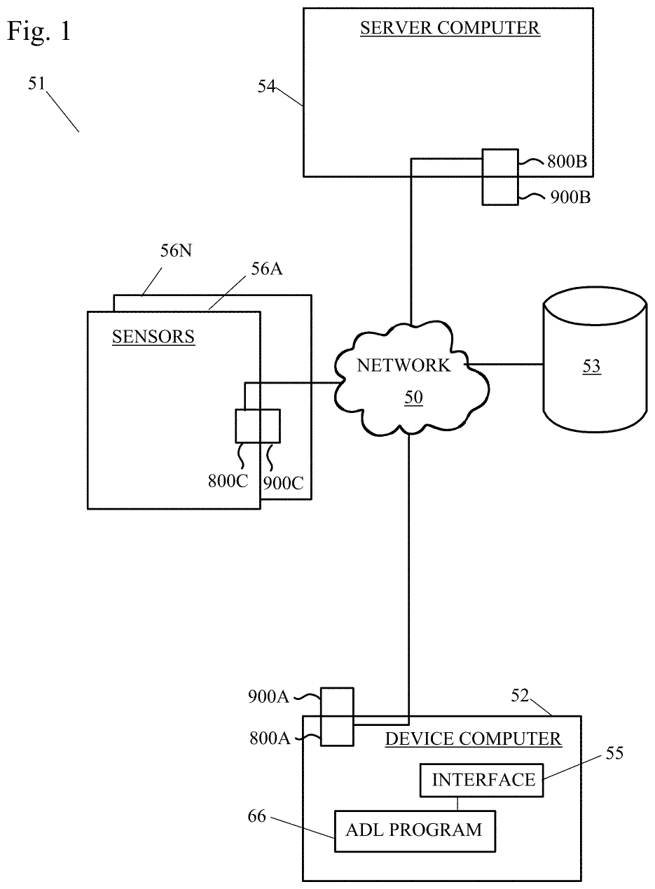

FIG. 1 is an exemplary diagram of a possible data processing environment provided in which illustrative embodiments may be implemented. It should be appreciated that FIG. 1 is only exemplary and is not intended to assert or imply any limitation with regard to the environments in which different embodiments may be implemented. Many modifications to the depicted environments may be made.

Referring to FIG. 1, network data processing system 51 is a network of computers in which illustrative embodiments may be implemented. Network data processing system 51 contains network 50, which is the medium used to provide communication links between various devices and computers connected together within network data processing system 51. Network 50 may include connections, such as wire, wireless communication links, or fiber optic cables.

In the depicted example, device computer 52, a repository 53, a plurality of sensors 56a-56n and a server computer 54 connect to network 50. In other exemplary embodiments, network data processing system 51 may include additional client or device computers, sensors, storage devices or repositories, server computers, and other devices not shown.

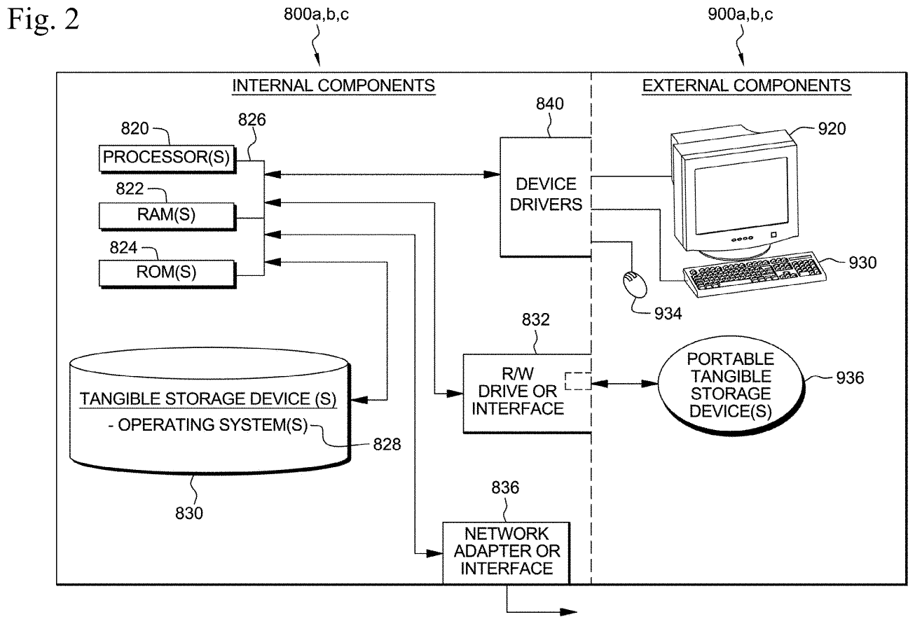

The device computer 52 may contain an interface 55, which may accept commands and data entry from a user or a plurality of sensors 56a-56n present within a smart environment. The commands may be regarding input to execute specific actions relative to devices present within a smart environment. The interface can be, for example, a command line interface, a graphical user interface (GUI), a natural user interface (NUI) or a touch user interface (TUI). The device computer 52 preferably includes an activity of daily living (ADL) program 66. The ADL program 66 can monitor and detect ADLs/IADLs, determine outlier ADLs and construct threshold based ADL temporal profiles. While not shown, it may be desirable to have the ADL program 66 be present on the server computer 54. The device computer 52 includes a set of internal components 800a and a set of external components 900a, further illustrated in FIG. 2.

Server computer 54 includes a set of internal components 800b and a set of external components 900b illustrated in FIG. 2. In the depicted example, server computer 54 provides information, such as boot files, operating system images, and applications to the device computer 52. Server computer 54 can compute the information locally or extract the information from other computers on network 50. The server computer 54 may contain the ADL program 66.

Sensors 56a-56n includes a set of internal components 800c and a set of external components 900c illustrated in FIG. 2. The sensors can sense various metrics associated with a user or a device of a user's environment. The sensors 56a-56n can briefly store data associated with the metrics and send the data via the network 50 to either the device computer 52 acting as a central processing hub or directly to the server computer 54. The metrics preferably include data associated with a timestamp.

Repository 53 preferably includes defined rules for activities of daily living and rules defining outlier conditions relative to the ADLs and IADLs. The repository can additionally store sensor data and threshold based ADL and IADL temporal profiles.

Program code and programs such as ADL program 66 may be stored on at least one of one or more computer-readable tangible storage devices 830 shown in FIG. 2, on at least one of one or more portable computer-readable tangible storage devices 936 as shown in FIG. 2, or on storage unit 53 connected to network 50, or may be downloaded to a device computer 52 or server computer 54, for use. For example, program code and programs such as ADL program 66 may be stored on at least one of one or more storage devices 830 on server computer 54 and downloaded to device computer 52 over network 50 for use. Alternatively, server computer 54 can be a web server, and the program code, and programs such as ADL program 66 may be stored on at least one of the one or more storage devices 830 on server computer 54 and accessed device computer 52. In other exemplary embodiments, the program code, and programs such as ADL program 66 may be stored on at least one of one or more computer-readable storage devices 830 on device computer 52 or distributed between two or more servers.

In the depicted example, network data processing system 51 is the Internet with network 50 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, network data processing system 51 also may be implemented as a number of different types of networks, such as, for example, an intranet, local area network (LAN), or a wide area network (WAN). FIG. 1 is intended as an example, and not as an architectural limitation, for the different illustrative embodiments.

FIG. 2 illustrates internal and external components of a device computer 52, sensors 56a-56n and server computer 54 in which illustrative embodiments may be implemented. In FIG. 2, a device computer 52, sensors 56a-56n, and a server computer 54 include respective sets of internal components 800a, 800b, 800c and external components 900a, 900b, 900c. Each of the sets of internal components 800a, 800b, 800c includes one or more processors 820, one or more computer-readable RAMs 822 and one or more computer-readable ROMs 824 on one or more buses 826, and on one or more operating systems 828 and one or more computer-readable tangible storage devices 830. The one or more operating systems 828 and ADL program 66 are stored on one or more of the computer-readable tangible storage devices 830 for execution by one or more of the processors 820 via one or more of the RAMs 822 (which typically include cache memory). In the embodiment illustrated in FIG. 2, each of the computer-readable tangible storage devices 830 is a magnetic disk storage device of an internal hard drive. Alternatively, each of the computer-readable tangible storage devices 830 is a semiconductor storage device such as ROM 824, EPROM, flash memory or any other computer-readable tangible storage device that can store a computer program and digital information.

Each set of internal components 800a, 800b, 800c also include a R/W drive or interface 832 to read from and write to one or more portable computer-readable tangible storage devices 936 such as a CD-ROM, DVD, memory stick, magnetic tape, magnetic disk, optical disk or semiconductor storage device. ADL program 66 can be stored on one or more of the portable computer-readable tangible storage devices 936, read via R/W drive or interface 832 and loaded into hard drive 830.

Each set of internal components 800a, 800b, 800c can also include a network adapter or interface 836 such as a TCP/IP adapter card. ADL program 66 can be downloaded to the device computer 52 and server computer 54 from an external computer via a network (for example, the Internet, a local area network or other, wide area network) and network adapter or interface 836. From the network adapter or interface 836, ADL program 66 is loaded into hard drive 830. ADL program 66 can be downloaded to the server computer 54 from an external computer via a network (for example, the Internet, a local area network or other, wide area network) and network adapter or interface 836. From the network adapter or interface 836, ADL program 66 is loaded into hard drive 830. The network may comprise copper wires, optical fibers, wireless transmission, routers, firewalls, switches, gateway computers and/or edge servers.

Each of the sets of external components 900a, 900b, 900c can include a computer display monitor 920, a keyboard 930, and a computer mouse 934. Each of the sets of internal components 800a, 800b, 800c also includes device drivers 840 to interface to computer display monitor 920, keyboard 930 and computer mouse 934. The device drivers 840, R/W drive or interface 832 and network adapter or interface 836 comprise hardware and software (stored in storage device 830 and/or ROM 824).

ADL program 66 can be written in various programming languages including low-level, high-level, object-oriented or non object-oriented languages. Alternatively, the functions of an ADL program 66 can be implemented in whole or in part by computer circuits and other hardware (not shown).

Activities of daily living (ADL) refer to people's daily self-care activities. Basic ADLs include, but are not limited to: self-feeding, walking (ambulating), dressing and grooming, bathing, toileting, and transferring (moving from one body position to another).

Instrumental Activities of Daily Living (IADL) are activities performed by an individual on a day to day basis that are not essential to basic self-care and independent living, but add quality to the way of life. Examples of IADLs are: care of others (including selecting and supervising caregivers), care of pets; child rearing; communication management; community mobility; financial management; health management and maintenance; home establishment and maintenance; meal preparation and cleanup (cooking); religious observances; safety procedures and emergency responses; and shopping.

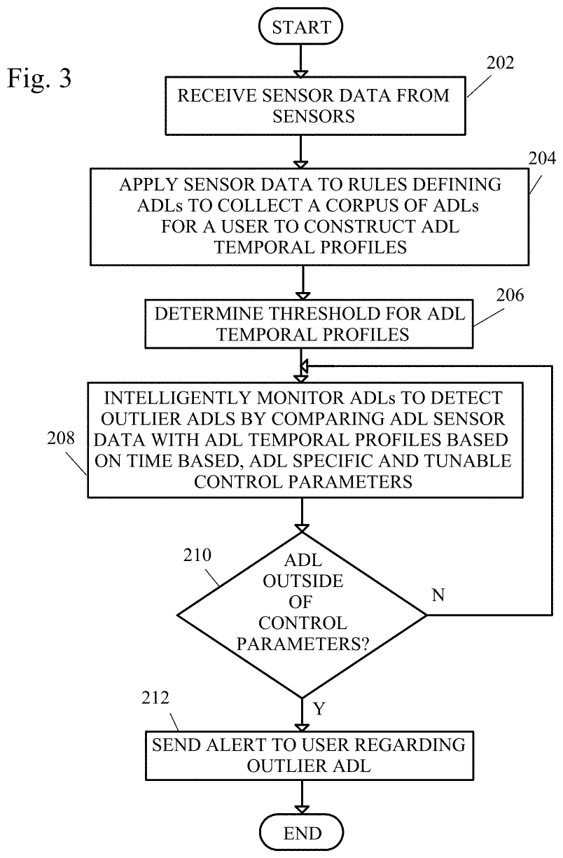

FIG. 3 is flow diagram of a method of determining outlier ADLs/IADLs and performing automatic actions in coordination with user inputs in smart environments.

In a first step, the ADL program 66 receives sensor data from sensors 56a-56n within a user's environment (step 202).

The ADL program 66 applies the sensor data received to rules defining ADLs/IADLs to collect a corpus of ADLs/IADLs including at least a start time of the activity, a stop time of the activity (to determine time spent completing activity for a user), and frequency of the activity for a user to construct ADL temporal profiles (step 204). The corpus and/or the rules can additionally include what sensors define the ADL/IADL. The ADL program 66 may also indirectly derive ADLs/IADLs by first identifying the existence of other ADLs from the sensor data received, and then deriving the presence of other ADLs due to the existence of already identified ADLs.

The threshold based ADL/IADL temporal profile is constructed by collected historic sensor data in a user's environment associated with the user. Using the ADL/IADL rules and the historic sensor data, a set of all of the ADLs/IADLs being performed by the user are inferred and a threshold is set. For each ADL/IADL, all data corresponding to a start time of the ADL/IADL is collected. Small time intervals are constructed that contain a certain fraction of the start time stamps that exceed the threshold. A frequency in which the ADLs/IADLs take place is determined by analyzing the start time stamps. The data corresponding to the amount of time taken by the ADL/IADL to complete is collected, and the usual time taken by the ADL/IADL can be calculated. Therefore, the threshold based ADL/IADL temporal profile maintains at least a distribution of start times, end times, average time taken for a user to complete an activity, tunable control parameters, and other inputs.

Values for the ADL/IADL specific parameters as well as other tunable control parameters can be set. Examples of tunable control parameters include: monitoring frequency, actions to perform, inputs to additionally consider, activation, and input to identify the possible exceptions during the execution of the ADL/IADL.

The ADL/IADL temporal profiles can be prioritized into categories: high, mid and low. The ADL/IADL temporal profiles can be applied to only certain priority levels such as “low”. Association of the priority to the ADL/IADL could be done explicitly by the user, or automatically by the smart home central processing hub by considering the importance and criticality of the ADL/IADL from the historical sensor data. By first only applying the temporal profiles to low priority ADL/IADL, it is possible capture learnings in terms of the user behavior, implications of the outlier handling methods, etc. This learning can later be used to improve the quality of the temporal profile model while constructing the temporal profiles to mid/high priority ADL/IADLs. Thus, it helps in improving the effectiveness of the construction of temporal profile generation for ADL/IADLs, as opposed to not having such a categorization in set of the ADL/IADLs of interest.

It should be noted that construction of initial threshold based ADL/IADL temporal profiles may take place as an off-line process. The threshold based ADL/IADL temporal profiles may be dynamically maintained and updated using machine learning based models.

The ADL program 66 then constructs thresholds based on the ADL/IADL temporal profiles associated with the ADLs/IADLs for the user and activates tunable control parameters with the ADL/IADL temporal profiles (step 206).

The tunable control parameters can be set to 1 to “activate” for a particular ADL/IADL temporal profile, otherwise the control parameter can be set to 0. If the value of the tunable “active” control parameter is set to 0, the ADL/IADL execution time windows are set as unbounded. If the value of the tunable “active” control parameter is set to 1, ADL/IADL execution time windows will be learned using machine learning techniques based on historical execution times and user specific parameters and will be time bounded. The time window bounds on the ADLs/IADLs are flexible.

An example of a threshold based IADL temporal profile for a “cooking” IADL can be set up to include the following parameters:

1) Time Based Parameters:
Start time distribution
End time distribution
Average total time taken

2) Counting Based Parameter
average frequency of cooking IADL in a day

3) IADL Specific Parameter
Smoke level parameter

4) Tunable Control Parameters
Activate (Y/N)
Action to Perform (No action, inform user, stop)
Monitor continuously or periodically
Inputs from other devices

Therefore, the cooking threshold based IADL temporal profile is:
Name: Cooking—Breakfast
Usual Time Taken to Completion: 10 minutes (with medium confidence)
Threshold: 14%
Start Time Distribution: [9:00-9:30] 75% of the time, [9:30-9:55] 25% of the time
Frequency: Repeats Daily
Smoke Level: Low
Activate: Yes
Input from Stove
Action to Perform: Inform User and possible turn off stove based on response from the User

An example of a threshold based ADL temporal profile for bathing is as follows:
Name: Bathing
Usual Time Taken to Completion: 15 minutes (with high confidence)
Threshold: 10%
Start Time Distribution: [9:00-9:30] 55% of the time, [9:30-9:55] 40% of the time
Frequency: Repeats Daily
Average Consumption of Water: 30 gallons
Activate: Yes
Action to Perform: Inform User

Once the threshold based temporal ADL/IADL profiles are set, the ADL program 66 selectively and intelligently monitors ADLs/IADLs to detect outlier ADLs by comparing sensor data to ADL/IADL temporal profiles based on time based, ADL/IADL specific, and tunable control parameters to determine outliers without explicitly checking status of each and every sensor within the environment (step 208).

An outlier is data, events or observations which do not conform to an expected pattern (temporal profile). The type of outlier may also be detected as well as whether the outlier is an exception. It should be noted that sensor data will only be collected during distribution times for a specific ADL/IADL. Outlier behavior is considered present if either the time taken by the ADL/IADL exceeds a typical time of execution (Type 1 outlier) or the ADL/IADL is not carried out after passing a typical time of execution (Type 2 outlier). An exception is an outlier of data that corresponds to an event which disrupts the IADL/ADL. In an embodiment of the present invention, for detected exceptions associated with a given ADL/IADL, predefined handling mechanisms may be used, for example calling the authorities. In a preferred embodiment, outlier behaviors which are not exceptions only trigger an alert to the user.

If the ADL/IADL is outside of the control parameters (step 210), an alert is sent to the user regarding the outlier ADL/IADL (step 212). The alert preferably contains the current state of the ongoing ADLs that exhibit exceptions (outside of the control parameters) and seeks appropriate user inputs to take specific action by a hub, such as device computer 52 of the smart environment. The user may also include other user such as family members, caregivers and/or a monitoring service.

If the ADL/IADL is outside of control parameters and is a Type 1 outlier, the alert is sent to user after completion of the usual time for the ADL/IADL. For example, of a user started the stove for cooking and left for an appointment without attending that IADL completely and due to inappropriate ingredients, additional smoke was detected, an alert can be sent to the user's mobile device seeking input regarding whether to take automatic action to end the IADL. If the user responds to end the IADL, the ADL program 66 executes an ending to the IADL, for example by switching power off to the stove. If the user does not respond within a specified time interval, the ADL program 66 can execute a default action for that ADL/IADL. This default behavior could have been chosen due to the historical observations by the central processing hub pertaining to a given user.

In another example, utilizing the IADL of cooking using the parameters described above, the smoke level parameter can be used to indicate the amount of smoke that is generated during normal historical “cooking” activity. If a user forgets to add water to the rice after putting the rice in the microwave and the rice turns black and generates smoke, the amount of smoke generated may be outside of the control parameters, (e.g more smoke than normally present during cooking), and an action, such as alerting the user or automatically stopping the microwave can be executed.

In yet another example, if the IADL is “entering the house”, the threshold based IADL temporal profile can contain the following parameters of: time base parameters, average number of time the activity of “entering the house” occurs in a day, whether the person is authorized, and other tunable parameters. Whenever this IADL occurs, the value of the “authorized parameter” indicates whether the entered person is authorized to enter or not. To determine whether the person is authorized, a wearable device may be registered with the ADL program 66. Through sensors 56a-56n present within the smart environment of the house, the wearable device can be detected and the user authenticated to determine whether they are authorized. The “authorized” parameter is set to 1 for being an authorized person and 0 for unauthorized. In this case, there can be exceptions that are not outliers. If an authorized user enters the house outside of normal times (e.g. son visiting a user past 8 PM), the entering of the house is an outlier. If an unauthorized user (e.g. thief) enters the house at any time, the entering action detected is considered an exception and appropriate action can be taken.

If the ADL/IADL is outside of control parameters and is a Type 2 outlier where the ADL/IADL is not carried out after passing a typical time of execution, an alert is sent about the ADL/IADL, for example to a mobile device of the user or another user. The Type 2 outlier is identified by comparing the control parameters to a start time distribution for all ADLs/IADLs associated with the user. An example of an alert message sent to the user may be “Water Pumping Motor is not switched on”. The alert message preferably includes an interface to receive the user's commands with suggested actions for the user to take regarding the ADL/IADL. For example, the user can respond to take the action and the ADL program 66 initiates an automatic start of the ADL (e.g. turning on the water pumping motor). If the user does not respond to take action or does not initiate the ADL, the ADL program 66 can stop monitoring the ADL/IADL for a given time period. In another example of user suggested actions, if the user left the smart environment prior to completion of the ADL/IADL the ADL program 66 can turn off specific appliances.

By using threshold based ADL temporal profiles containing a tunable control parameter of “active”, the ADL program 66 does not have to track non-active devices at a given time. Instead, sensors associated with a given ADL/IADL are tracked during the start time distribution without polling each and every sensor in the entire smart environment. Furthermore, command execution times in asynchronous computing environments are reduced, aiding users which may be displaying multiple outlier behaviors relative to an ADL in time critical scenarios.

If a user left the workplace late and didn't cook the dinner between the usual time period 5:00 PM-6:30 PM, an alert can be sent to the user's mobile device seeking input regarding whether to take automatic action to start the IADL. If the user responds to start the IADL, the ADL program 66 executes to start the IADL, for example by switching power on the stove. If the user does not respond within a specified time interval, the ADL program 66 can execute a default action for that ADL/IADL.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method of determining outlier activities of a user relative to daily living activities or instrumental daily living activities in a smart environment, the smart environment comprising a plurality of sensors in communication with a computer, the method comprising the steps of:
the computer receiving sensor data from the plurality of sensors in the smart environment;
the computer applying the sensor data to rules defining activities of daily living and instrumental activities of daily living to collect a corpus of activities of daily living and instrumental activities of daily living of the user;
the computer constructing an activity of daily living and instrumental activity of daily living temporal profile based on the corpus of activities associated with the user;
the computer setting a threshold for each activity of daily living and instrumental activity of daily living temporal profile associated with the user;
the computer activating and monitoring the plurality of sensors associated with activity of daily living and instrumental activity of daily living during a distribution of start times associated the activity of daily living and instrumental activity of daily living temporal profiles to determine outliers and exceptions of the activities of daily living and instrumental activities of daily living associated with the user above the threshold;
the computer, for each outlier, sending an alert to at least the user regarding the outlier with associated actions within the smart environment; and
the computer receiving chosen associated actions from the user and implementing the associated actions within the smart environment.

2. The method of claim 1, wherein the activity of daily living and instrumental activity of daily living temporal profile comprises: time based parameters including start time distribution, end time distribution and average total time taken; counting based parameters of an average frequency in which the daily living activity or instrumental daily living activity takes place within a given time period; activity specific parameters; and tunable control parameters including actions to perform, monitoring frequency, and activation.

3. The method of claim 1, wherein the associated actions discontinue monitoring of the activities of daily living or instrumental activities of daily living.

4. The method of claim 1, wherein the associated actions, through the plurality of sensors, turn on or off devices of the smart environment.

5. The method of claim 1, wherein the outlier is an activity of daily living or instrumental activity of daily living which exceeds the threshold associated with the average total time for the activity of daily living or instrumental activity of daily living within the activity of daily living and instrumental activity of daily living temporal profile.

6. The method of claim 1, wherein the outlier is a lack of an activity of daily living or instrumental activity of daily living during the distribution of start times associated the activity of daily living and instrumental activity within the activity of daily living and instrumental activity of daily living temporal profile.

7. The method of claim 1, wherein the alert is send to at least one user selected from the group consisting of: a family member of the user; a caregiver; and a monitoring service.

8. A computer program product for determining outlier activities of a user relative to daily living activities or instrumental daily living activities in a smart environment, the smart environment comprising a plurality of sensors in communication with a computer comprising at least one processor, one or more memories, one or more computer readable storage media, the computer program product comprising a computer readable storage medium having program instructions embodied therewith, the program instructions executable by the computer to perform a method comprising:
receiving, by the computer, sensor data from the plurality of sensors in the smart environment;
applying, by the computer, the sensor data to rules defining activities of daily living and instrumental activities of daily living to collect a corpus of activities of daily living and instrumental activities of daily living of the user;
constructing, by the computer, an activity of daily living and instrumental activity of daily living temporal profile based on the corpus of activities associated with the user;
setting, by the computer, a threshold for each activity of daily living and instrumental activity of daily living temporal profile associated with the user;
activating and monitoring, by the computer, the plurality of sensors associated with activity of daily living and instrumental activity of daily living during a distribution of start times associated the activity of daily living and instrumental activity of daily living temporal profiles to determine outliers and exceptions of the activities of daily living and instrumental activities of daily living associated with the user above the threshold;
for each outlier, sending, by the computer, an alert to at least the user regarding the outlier with associated actions within the smart environment;
for each exception, implementing the associated actions within the smart environment based on predefined actions; and
receiving, by the computer, chosen associated actions from the user and implementing the associated actions within the smart environment.

9. The computer program product of claim 8, wherein the activity of daily living and instrumental activity of daily living temporal profile comprises: time based parameters including start time distribution, end time distribution and average total time taken; counting based parameters of an average frequency in which the daily living activity or instrumental daily living activity takes place within a given time period; activity specific parameters; and tunable control parameters including actions to perform, monitoring frequency, and activation.

10. The computer program product of claim 8, wherein the associated actions discontinue monitoring of the activities of daily living or instrumental activities of daily living.

11. The computer program product of claim 8, wherein the associated actions, through the plurality of sensors, turn on or off devices of the smart environment.

12. The computer program product of claim 8 wherein the outlier is an activity of daily living or instrumental activity of daily living which exceeds the threshold associated with the average total time for the activity of daily living or instrumental activity of daily living within the activity of daily living and instrumental activity of daily living temporal profile.

13. The computer program product of claim 8, wherein the outlier is a lack of an activity of daily living or instrumental activity of daily living during the distribution of start times associated the activity of daily living and instrumental activity within the activity of daily living and instrumental activity of daily living temporal profile.

14. The computer program product of claim 8, wherein the alert is send to at least one user selected from the group consisting of: a family member of the user; a caregiver; and a monitoring service.

15. A computer system for determining outlier activities of a user relative to daily living activities or instrumental daily living activities in a smart environment, the smart environment comprising a plurality of sensors in communication with a computer, the computer system comprising the computer comprising at least one processor, one or more memories, one or more computer readable storage media having program instructions executable by the computer to perform the program instructions comprising:
receiving, by the computer, sensor data from the plurality of sensors in the smart environment;
applying, by the computer, the sensor data to rules defining activities of daily living and instrumental activities of daily living to collect a corpus of activities of daily living and instrumental activities of daily living of the user;
constructing, by the computer, an activity of daily living and instrumental activity of daily living temporal profile based on the corpus of activities associated with the user;
setting, by the computer, a threshold for each activity of daily living and instrumental activity of daily living temporal profile associated with the user;
activating and monitoring, by the computer, the plurality of sensors associated with activity of daily living and instrumental activity of daily living during a distribution of start times associated the activity of daily living and instrumental activity of daily living temporal profiles to determine outliers and exceptions of the activities of daily living and instrumental activities of daily living associated with the user above the threshold;
for each outlier, sending, by the computer, an alert to at least the user regarding the outlier with associated actions within the smart environment; and
receiving, by the computer, chosen associated actions from the user and implementing the associated actions within the smart environment.

16. The computer system of claim 15, wherein the activity of daily living and instrumental activity of daily living temporal profile comprises: time based parameters including start time distribution, end time distribution and average total time taken; counting based parameters of an average frequency in which the daily living activity or instrumental daily living activity takes place within a given time period; activity specific parameters; and tunable control parameters including actions to perform, monitoring frequency, and activation.

17. The computer system of claim 15, wherein the associated actions discontinue monitoring of the activities of daily living or instrumental activities of daily living.

18. The computer system of claim 15, wherein the associated actions, through the plurality of sensors, turn on or off devices of the smart environment.

19. The computer system of claim 15, wherein the outlier is an activity of daily living or instrumental activity of daily living which exceeds the threshold associated with the average total time for the activity of daily living or instrumental activity of daily living within the activity of daily living and instrumental activity of daily living temporal profile.

20. The computer system of claim 15, wherein the alert is send to at least one user selected from the group consisting of: a family member of the user; a caregiver; and a monitoring service.
